(19) United States
(12) Patent Application Publication
(10) Pub. No.: US 2010/0016883 A1
(43) Pub. Date: Jan. 21, 2010

(54) DOUBLE GRASPER AND TRIGGER CONTROL MECHANISM

(76) Inventor: George C. Christoudias, Teaneck, NJ (US)

Correspondence Address: RICHARD A. JOEL ESQ., 496 KINDERKAMACK ROAD, ORADELL, NJ, 07649, US

(21) Appl. No.: 12/175,943

(22) Filed: Jul. 18, 2008

(51) Int. Cl. A61B 17/00; A61B 1/00

(52) U.S. Cl. 606/205; 600/118

(57) ABSTRACT

This invention comprises an instrument with a unique trigger mechanism for approximating any tissues that can physically reach each other during a performance of any laparoscopic procedure. The user friendly double grasper comprises an elongated cannula having visually coded jaws at one end which cooperate with an intermediate member. The visually coded jaws are connected to control means within the cannula which are coupled to tacile coded control triggers mounted to a longitudinal handle. Each trigger is spring loaded to passively close a particular jaw after manually opening the jaw. Both triggers may be operated individually or simultaneously with the same operating digit. The triggers include a dimple on one trigger and a bubble on the other trigger so that an operator may recognize which trigger corresponds to which jaw and thus control the jaws by feel. The jaws are also coded for visual recognition with different color or letter.

A double grasper with a new and improved trigger control mechanism comprises a narrow elongated cannula having a pair of jaws at one end which each cooperate with a central plate to secure tissues and permit a surgeon to manipulate the tissues in a laparoscopic procedure. The jaws are mounted on one end of an elongated cannula which includes two spring actuated user friendly triggers at the other end mounted on a longitudinal handle. Each trigger controls a specific jaw and is manipulated to open the jaw but passively closes without manipulation. The triggers may be operated by a single digit simultaneously or individually.

The present invention permits approximating any tissues that can physically reach each other during the performance of a laparoscopic procedure. With the flaps held together by the present invention, the suturing or stapling of the tissue flaps is greatly facilitated. A double grasper with two independent jaws and a unique trigger arrangement is used to sequentially grasp both tissue areas to be approximated against the central protruding plate and hold the tissue for stapling or suturing.

This invention represents an improvement over the Maritsa Tissue Approximator and Method of Using Same disclosed in U.S. Pat. No. 5,403,332 to applicant. This patent comprises an elongated cylinder which is divided into two elongated compartments by a central plate extends outwardly from the bottom of the cylinder. Independently controlled jaws close separately over the fixed plate to move a tissue part into approximation with a second tissue part to facilitate fixation of two tissue parts together. The patent also includes the graspers with different jaw configurations such as the Babcock, Ellis, Bucktooth, etc. that are currently used in the field.

More particularly, this application represents an improvement over applicant's pending application on an Atraumatic Single or Double Tissue Grasper Ser. No. 11/554,271 filed Oct. 30, 2006. As with most graspers, the jaws must be positively activated to open and deactivate to close. Also of interest are applicant's U.S. Pat. Nos. 5,817,121 and 6,391,046 on the Christoudias endodissector.

The double grasper comprising this invention relates to laparoscopic surgery and in particular a method employed in procedures involving tissue approximation such as nissen fundoplication, intestinal anastomosis, etc., an apparatus and method employed in laparoscopic herniography, wherein the laparoscopic tissue is approximated by the apparatus. This unique instrument permits the atraumatic manipulation of tissues in the abdominal cavity while securely and independently grasping and approximating or folding two different areas of tissues. The double grasper comprises a narrow cylinder or cannula having control rods extending longitudinally therewithin. The control end of the double grasper includes a handle and two independently operating spring loaded triggers mounted thereon with a control wire coupled to each trigger. The triggers are spring loaded and may be operated independently or together by one's thumb. The pivotal jaws are each connected at the other end of the control rod of a specific trigger. This allows each jaw to open and close independently of each other against a fixed intermediate plate. More importantly, the triggers are user friendly wherein the jaws open on manipulation of a particular trigger but then close automatically on release of the trigger. The triggers include means for tacile recognition of what jaw is being operated while the jaws include color coding or letter for visual recognition

In operation, the separated edges of a tissue can be approximated one at a time bringing them together in preparation for suturing and/or stapling to each other. Specifically, a distal peritoneal flap is engaged between a first jaw and the fixed plate by opening and releasing the trigger thus closing the jaw over the fixed plate with the tissue therebetween. The head of the instrument is then moved to the proximal flap with the jaws closed the second jaw recognized by the color or letter code is opened by depressing the second trigger recognized by the tacile code to correspond to the said jaw and then released to grasp the proximal flap. Thus the instrument approximates and holds the two flaps together for suturing or stapling. The subject tissue may be readily moved to the left or the right pulling the proximal and distal flaps together for stapling with ease. The instrument is simple and easy to use.

Accordingly, an object of this invention is to provide a new and improved trigger mechanism in apparatus for approximating tissue in laparoscopic surgery.

Another object of this invention is to provide a new and improved double grasper with a unique trigger arrangement for approximating tissue in laparoscopic surgery so that the tissue may be readily and expeditiously sutured or stapled during an operation.

A further object of this invention is to provide a new and improved double grasper which includes independently operated jaws which are positively opened with a single digit and then close passively to grasp certain tissue against a central plate under control of spring loaded triggers mounted to the handle.

A more specific object of this invention is to provide a new and improved instrument for laparoscopic surgery specifically a double grasper for approximating tissues which can physically reach each other by using independently operated jaws controlled by user friendly spring loaded triggers which actively open but passively close, mounted to a handle which include a dimple on one trigger and a bubble on the other for tacile recognition of the jaw being operated and color or letter code of the jaws for visual recognition of the jaws, allowing identification of which trigger corresponds to which jaw.

The above and other objects and advantages of the present invention may be more clearly seen when viewed in conjunction with the accompanying drawings wherein:

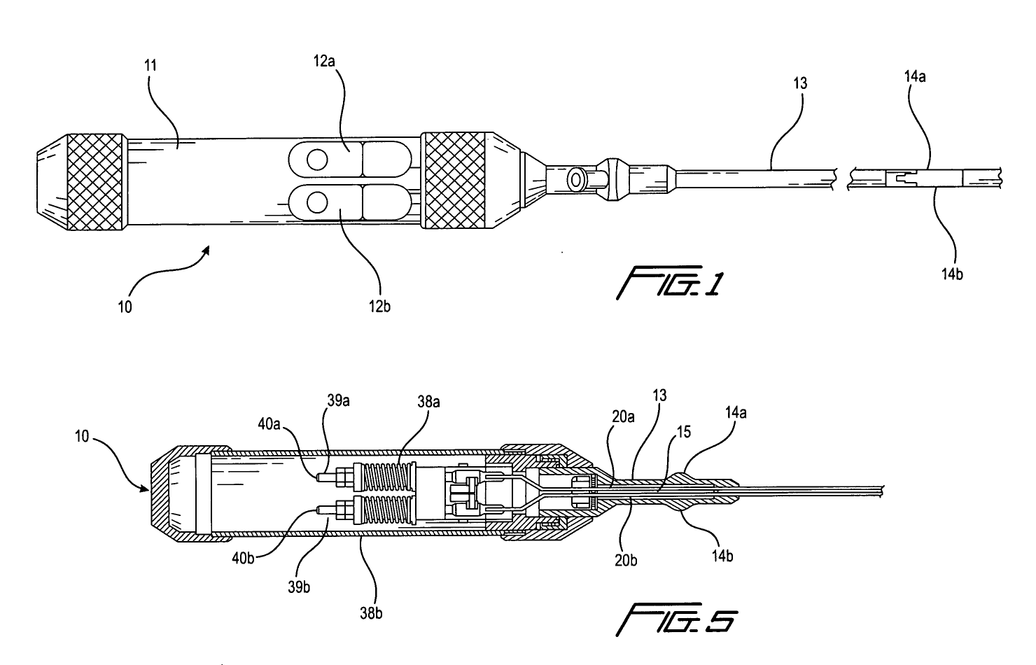

FIG. 1 is a top view of the invention;

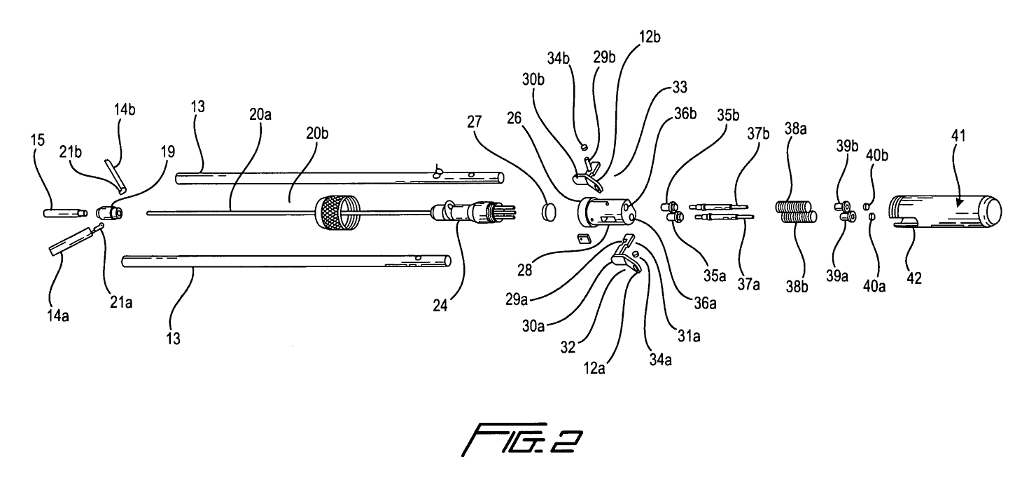

FIG. 2 is an exploded view of the invention;

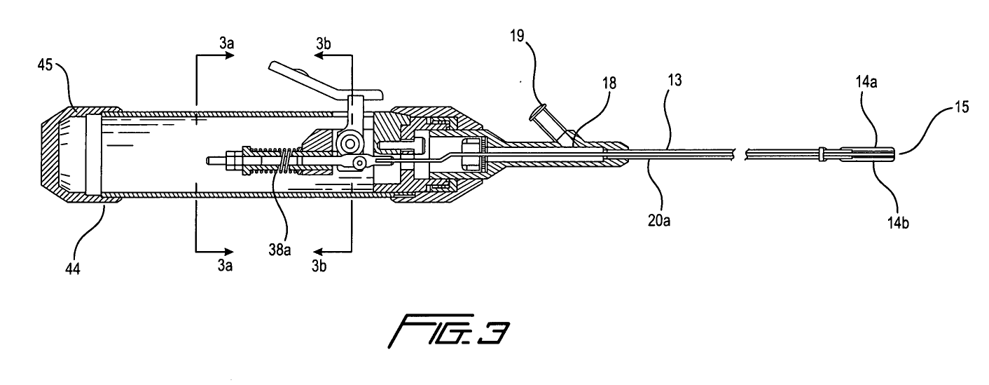

FIG. 3 is a side cross-sectional view of FIG. 1;

FIG. 3a is an enlarged cross-sectional view taken along the lines 3a-3a of FIG. 3;

FIG. 3b is an enlarged cross-sectional view taken along the lines 3b-3b of FIG. 3;

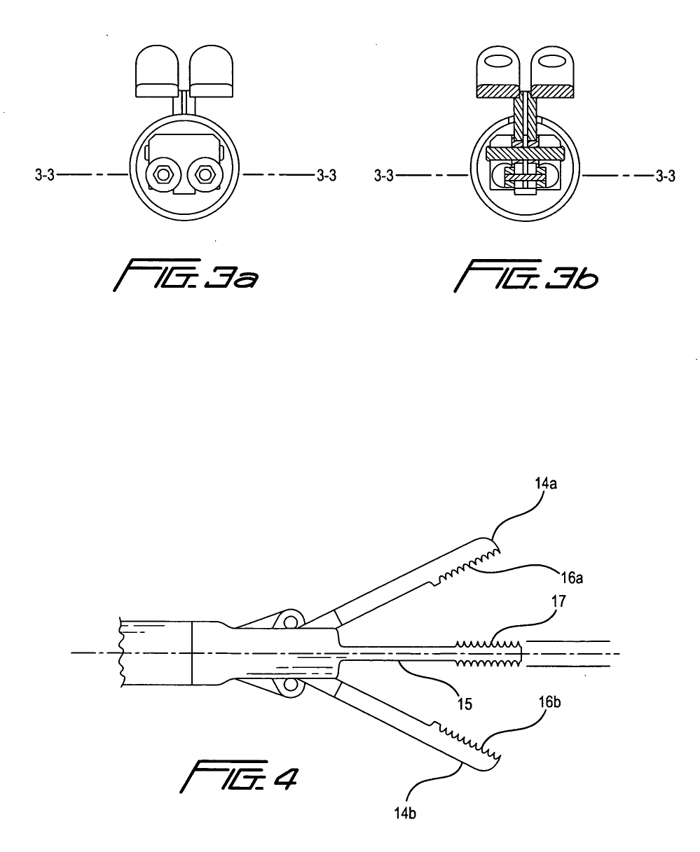

FIG. 4 is an enlarged view of the jaws of FIG. 1 in operation; and

FIG. 5 is a bottom view in cross-section of the subject invention shown in FIG. 1.

Referring now to the drawings, this application relates to instruments for laparoscopic surgery and in particular a double grasper 10 known as the Christoudias Approximator. The instrument 10 comprises a handle 11 having pair of triggers 12a, 12b mounted thereto, an elongated cannula 13 extending longitudinally along the hand axis and being coupled to the handle 11 at one end and having a pair of grasping jaws 14a, 14b at the other end. The jaws 14a, 14b are each controlled by a separate trigger 12a, 12b said triggers 12a, 12b being physically adjacent to each other. The jaw 14a grasps tissue against a central plate 15 so that the tissue may be moved or manipulated to a point where the second jaw 14b grasps another portion of tissue to bring the two tissues together for suturing, etc.

Importantly, the triggers 12a and 12b open the jaws 14a and 14b respectively but the jaws 14a, 14b close automatically when pressure is released on the triggers 12a, 12b. The trigger design is more user friendly than the prior art where active manipulation is required to both open and close the jaws 14a, 14b.

The jaws 14a, 14b passively close without manipulation due to the action springs 38a, 38b.

The operation of the jaws 14a, 14b in an open position is shown in FIG. 4 with each jaw having a plurality of teeth 16 along the internal face for cooperating with the teeth 17 on the central plate 15. The central plate 15 may be covered with gauze if desired. The elongated cannula 13 also includes a clean-out aperture 18 with a plug 19 in at the other end of the cannula 13.

FIG. 2 shows an exploded view of the invention wherein the jaws 14a, 14b each include a rear projecting portion 21a, 21b respectively mounted within an aperture 19 the stationary portion 18 for pivotal movement. A central plate 15 is also depicted which is engaged by the jaws 14a, 14b on opposite sides.

Control rods 20a, 20b are enclosed within a heat shrink layer 23 within an elongated cannula 22. The control rods 20a, 20b are mounted to respective jaws 14a, 14b at one end and are coupled to the respective triggers 12a, 12b at the other end. Pressing trigger 12a operates jaw 14a via control rod 20a and similarly control rod 20b with trigger 12b operates jaw 14b.

The control rods 20a, 20b extend through a nose cone 24 with a clean out plug 25. The nose cone 24 is threaded onto a hollow super structure 26 with an internal seal 27. The structure 26 includes an aperture 28 into which the trigger portions 31a, 31b are inserted. The triggers 12a, 12b each include an inclined upper portion 30a, 30b with a dimple 32 on one portion and a bubble 33 on the other portion so that the surgeon can recognize by tacile recognition what jaw 14a or 14b he is operating.

A trigger shaft 29a, 29b projects inwardly from each portion 31a, 31b and includes roller 34a, 34b at its end. Driver shaft guides 35a, 35b are mounted within apertures 36a, 36b in structure 26 and a drive shaft 37a, 37b is mounted within each guide 35a, 35b. Springs 38a, 38b are mounted about each shaft 37a, 37b and secured in place by a spring guide 39a, 39b and hex nut 40a, 40b. The springs 38a and 38b cause automatic closure of the jaws 14a, 14b when pressure is released on the particular trigger.

A hollow tube body 41 having a trigger slot 42 is mounted over the structure 26 and internal controls and nose cone 24 is threadingly mounted thereto. At the other end of the tube body 41 a knurled end portion 45 engages exterior threads 44.

FIG. 3a shows an enlarged rear cross-sectional view of the trigger assembly while FIG. 3b shows an enlarged front cross-sectional view of the trigger assembly.

FIG. 4 shows the jaws 14a and 14b of the double grasper 10 in an open position. The internal teeth 16a and 16b on the jaws 14a, 14b cooperate with the teeth 17 on the central plate 15. Alternatively, the central plate 15 and indeed the jaws 14a, 14b may be gauze covered.

In operation, the double grasper 10 is inserted through a standard laparoscopic port. The jaws 14a and 14b and cannula 13 are small enough, approximately 10 mm in diameter, to fit through the port. The surgeon manipulates the instrument 10 usually with one hand and operates the trigger 12a with one's thumb to grasp the desired tissue between a jaw 14a and the central plate 15 as pressure is released on the trigger 12a. Next the grasper 10 is moved to an adjacent tissue and the jaw 14b is opened using one's thumb with trigger 12b to grasp another tissue upon release so that the tissue held by the two jaws 14a, 14b and central plate 15 are in proximity for suturing, etc.

The operation is often conducted in a darkened room so that tacile recognition of what trigger 12a or 12b controls what jaw 14a or 14b is noted by the tacile recognition of the dimple 32 or bubble 33 on the triggers 12a, 12b. Both triggers 12a, 12b can be operated together so that the tissue can be readily released or grasped. As a further advantage, the double grasper 10 may be easily held and operated with one hand freeing the surgeon's hand for other purposes.

As shown in FIGS. 2 and 3, each trigger 12a, 12b is mounted to a vertical member 31a, 31b within the handle 11 which actuates a spring loaded member 37a, 37b coupled to the control rod 20a or 20b. The cannula 13 is held in place within the handle 11 by a front nut 23 which is threaded onto the handle 11 while the rear portion 24 of the handle 11 includes another threaded member 25 to cover the aperture 18 in the hollow handle 11.

One of the triggers 12a includes a dimple 31 and the other trigger 12b includes a bubble 32 for tactile recognition of the particular trigger during an operation. One of the jaws 14a is gold and the other jaw 14b is a silver metal for color recognition of the particular jaw or lettering may be used on the jaws 14a, 14b. The spring loaded feature of the triggers 12a, 12b permits faster closing of the jaw 14a, 14b without manipulation of the triggers 12a, and 12b. Further, the jaws 14a, 14b may be both activated together or singularly. Each jaw 14a, 14b is coupled by a wire 37a, 37b to a particular spring 38a, 38b. Pressing the trigger 12a, 12b downwardly compresses the spring 38a, 38b and opens the jaw 14a, 14b. When the trigger 12a, 12b is released; the jaw 14a, 14b closes automatically to grasps the tissue.

While the invention has been explained by a detailed description of certain specific embodiments, it is understood that various modifications and substitutions can be made in any of them within the scope of the appended claims that are intended also to include equivalents of such embodiments.

1. A double grasper for approximating tissues in laparoscopic surgery with one hand comprises:
a longitudinal handle having a first and second trigger mounted thereon adjacent to each other;
means for tactile recognition on each trigger and visual recognition for each jaw;
an elongated cannula having internal control means coupled at one end to each trigger and mounted within the handle and extending longitudinally outward therefrom;
a first jaw and a second jaw at the other end of the elongated cannula having a plate mounted between said jaws wherein each trigger controls a particular jaw to open and passively close to grasp tissue against the plate to approximate the tissue for further operations; and,
wherein the triggers may be operated by a single digit.

2. A double grasper in accordance with claim 1 wherein:
the jaws each include internal toothed edges; and,
the plate includes a first and a second side each having teeth to mesh with the teeth on the jaws.

3. A double grasper in accordance with claim 2 further including:
a clean-out port mounted on the handle.

4. A double grasper in accordance with claim 1 wherein:
the triggers include an operative upper portion and are each spring loaded, said springs being coupled to the control means to close the jaw upon release of the trigger;
the means for tactile recognition of the jaw being operated includes a dimple on the operative upper portion of one trigger; and,
includes a bubble on the operative portion of the other trigger for tactile recognition of the particular jaw being activated.

5. A double grasper in accordance with claim 1 wherein:
the triggers are situated in adjacent positions to each other and can be activated one at a time or both together opening the jaws one at a time or both simultaneously with the same operating digit (thumb).

6. A double grasper in accordance with claim 1 wherein:
the plate is gauze covered.

7. A double grasper in accordance with claim 1 wherein:
the control means comprises a first elongated rod connected at one end to a first pivotal jaw and a second elongated rod coupled at one end to a second pivotal jaw, said elongated rods being coupled to a particular trigger at their other end, said triggers being spring loaded to maintain the respective jaws in a normally closed position.

8. (canceled)

9. (canceled)

10. A double grasper in accordance with claim 5 wherein:
each trigger further includes a downwardly extending portion having a transverse shaft affixed thereto;
an aperture in said handle through which the downwardly extending trigger portions are positioned; and,
a drive shaft coupled to each trigger shaft and to a control rod within the handle and a coiled spring mounted about each shaft to bias said triggers to automatically close a particular jaw upon release of the particular trigger.

11. A double grasper in accordance with claim 1 wherein:
the jaws are color coded to provide visual recognition of the particular jaw corresponding to a particular trigger.

12. A double grasper in accordance with claim 1 wherein:
the jaws include lettering to provide visual recognition of the jaw being operated by a particular trigger.
